(19) United States
(12) Patent Application Publication
(10) Pub. No.: US 2023/0250445 A1
(43) Pub. Date: Aug. 10, 2023

(54) Spinach Plant Resistant to Downy Mildew and Novel Resistance Gene

(71) Applicant: Enza Zaden Beheer B.V., Enkhuizen (NL)

(72) Inventors: Faira Suidgeest, Enkhuizen (NL); Ilja Roobeek, Enkhuizen (NL); Jan Ane Dijkstra, Enkhuizen (NL)

(21) Appl. No.: 18/015,110

(22) PCT Filed: Jun. 29, 2021
(86) PCT No.: PCT/EP2021/067845

§ 371 Date: Jan. 9, 2023

(30) Foreign Application Priority Data
Jul. 10, 2020 (WO) PCT/EP2020/069608

(51) Int. Cl. C12N 15/82

(52) U.S. Cl. CPC C12N 15/8282

(57) ABSTRACT

The present invention relates to a spinach plant that is resistant to downy mildew caused by Peronospora farinosa. The present invention further relates to a resistance gene that confers resistance to downy mildew in spinach plants, and methods for obtaining a spinach plant that is resistant to downy mildew, and use of one or more markers for providing a spinach plant that is resistant to downy mildew.

### CROSS-REFERENCE TO RELATED APPLICATIONS

This application is the United States national phase of International Application No. PCT/EP2021/067845 filed Jun. 29, 2021, and claims priority to International Application No. PCT/EP2020/069608 filed Jul. 10, 2020, the disclosures of which are hereby incorporated by reference in their entirety.

The Sequence Listing associated with this application is filed in electronic format via EFS-Web and is hereby incorporated by reference into the specification in its entirety. The name of the text file containing the Sequence Listing is 2207798_ST25.txt. The size of the text file is 23,728 bytes, and the text file was created on Nov. 30, 2022.

### BACKGROUND OF THE INVENTION

### Field of the Invention

The present invention relates to a spinach plant that is resistant to downy mildew caused by Peronospora farinosa. The present invention further relates to a resistance gene that confers resistance to downy mildew in spinach plants, and methods for obtaining a spinach plant that is resistant to downy mildew, and use of one or more markers for providing a spinach plant that is resistant to downy mildew.

### Description of Related Art

Spinach (Spinacia oleracea) is an open field crop grown in many diverse environments. Spinach is a diploid crop that grows well in areas that have a cool, wet spring period, cool summers and dry autumns. Optimal soil conditions include well-drained soils and a pH above 6. Nowadays, spinach breeding mainly focusses on disease resistance (e.g. downy mildew), crop yield and improved nutritional value.

Plant breeding and screening activities help to select varieties in the main production regions, where local market adaptation and dynamic resistance are important factors to success. Spinach breeding programmes are developed that aim to provide for varieties for all market segments; the fresh (baby leaf) market, bunching market as well as frozen and canned products. Several specific varieties of spinach are available on the market, within the main types: smooth, savoyed and oriental types. The spinach market is growing rapidly worldwide and much of research is being performed to improve genetics of the spinach, for instance in relation to disease resistance and reducing the need for biochemicals or pesticides, and to improve both crop yield and crop quality. Another goal of the breeding programs is to provide for spinach varieties with broad resistance patterns to downy mildew caused by Peronospora farinosa, and ideally already taking future strains into account.

Downy mildew refers to several types of oomycete microbes that are parasites of plants. Downy mildew can originate from various species, but mainly of Peronospora, Plasmopara and Bremia. Downy mildew is a problem in many food crops, and in spinach caused by Peronospora farinosa sp. (Pfs), affecting the production of this crop worldwide. Downy mildew is one of the most problematic diseases in spinach. Spinach Downy mildew infection show symptoms of discoloured areas and irregular yellow patches on upper leaf surfaces in combination with white, grey or purple mould located on the other side of the leaf surface below. Lesions may eventually dry out and turn brown. Disease is spread from plant to plant by airborne spores.

Fungicides can be used to control Peronospora farinosa, but eventually Peronospora farinosa becomes immune to these chemicals, because over time the pathogen also acquires resistance to fungicides. In addition, the market wishes to reduce the use of such chemicals in the production of food crops. Therefore, it is of the utmost importance to find other methods to control Peronospora farinosa infection. Most preferably is to identify a resistance gene that gives broad resistance against Peronospora farinosa. Also resistance genes can be combined to achieve a broad scope and durable resistance against Peronospora farinosa. Therefore, identification of new resistance genes is a promising alternative.

Seventeen official races of Peronospora farinosa have been identified to date (Pfs1 to Pfs17) and characterization is based on qualitative disease reactions on a set of host differentials, an approach widely used to identify races of many plant pathogens. For spinach, the current set of differentials is comprised of new and old commercial hybrids as well as open-pollinated cultivars and breeding lines (NIL lines). In addition, the pathogen under pressure mutates to break down the disease resistance and new disease resistance in crops is needed to control infection. Especially in spinach the occurrence of resistant downy mildew is particularly complex as there are many different races, and new resistant downy mildew species, i.e. races that break current resistances emerge all the time. Breakthrough can occur as quick as within 4 to 6 months. The main problem is that the present spinach varieties on the market combining different resistances become very fast outdated as Peronospora farinosa quickly evolves new virulent races. With new races of downy mildew popping up in spinach over the last several years, it becomes increasingly more difficult to stay a step ahead of the devastating disease.

At present all known Peronospora farinosa resistance in spinach originate from (a combination of) resistant alleles present on a single locus (locus 1) in spinach. At present there is no single resistance gene available that provides full spectrum resistance to all races/physio's of Peronospora farinosa. Therefore, it is an advantage to combine or stack multiple resistance genes into a spinach plant, such that a plant is obtained that comprises multiple resistance genes and is resistant to all Peronospora farinosa physios, or at least is resistant to as many Peronospora farinosa physios as possible.

Considering the above, there is a need in the art to develop a more diverse and durable resistance in spinach and to provide spinach plants that are resistant to downy mildew caused by Peronospora farinosa and wherein these plants have a broad spectrum resistance against this pathogen that causes downy mildew. Furthermore, it is an object of present invention to provide a method to obtain such downy mildew resistant plants. There is a need for more diversity of alleles and/or another locus, so that more genetic variation can be achieved in commercial hybrids, making it harder for pathogens such as Peronospora farinosa to adapt. The broader the resistance of these alleles, the more effectively they can be used in the development of resistant plants.

### SUMMARY OF THE INVENTION

It is an object of the present invention, amongst other objects, to address the above need in the art. The object of present invention, amongst other objects, is met by the present invention as outlined in the appended claims.

Specifically, the above object, amongst other objects, is met, according to a first aspect, by the present invention by a spinach plant that is resistant to downy mildew caused by Peronospora farinosa (Pfs), wherein the spinach plant comprises in its genome a resistance gene comprising one or more mutations, wherein said resistance gene is associated with markers of SEQ ID No. 3 and SEQ ID No. 4 or SEQ ID No. 5 and SEQ ID No. 6. The resistance gene is also referred to herein as L3 gene.

According to a preferred embodiment, present invention relates to the spinach plant, wherein said resistance gene encodes for a protein having at least 85% sequence identity with SEQ ID No. 2, preferably at least 90%, more preferably at least 95%, even more preferably at least 98%, most preferably 99%. The identification of novel candidate dominant Pfs resistance genes are often also known as so called NBS-LLR genes, which are an important class of genes involved in disease resistance. The novel resistance gene was obtained by sequencing and gene mapping of Peronospora farinosa resistance genes on locus 3 on chromosome 1 in Spinach.

According to another preferred embodiment, the present invention relates to the spinach plant wherein said resistance gene comprises a coding sequence having at least 90% sequence identity with SEQ ID No. 1, preferably at least 95%, more preferably at least 99%, most preferably 99% sequence identity with SEQ ID No. 1.

According to a preferred embodiment, present invention relates to the spinach plant, wherein the plant is heterozygous or homozygous for the resistance gene comprising one or more mutations. Preferably the resistance gene is homozygously present in the genome of the plant.

According to another preferred embodiment, the present invention relates to the spinach plant wherein said plant is at least resistant to Peronospora farinosa races Pfs3 to Pfs17. The spinach of present invention plant is likely also resistant to Pfs1 and Pfs2.

According to yet another preferred embodiment, the present invention relates to the spinach plant wherein said resistance gene is obtainable from deposit number NCIMB 43624.

According to yet another preferred embodiment, the present invention related to the spinach plant, wherein the one or more mutations comprise deletions, insertions or substitutions in the coding sequence of SEQ ID No. 1. Mutations may be obtained via conventional breeding or by introducing the one or more mutations in the resistance gene by genome editing techniques, CRISPR Cas, or mutagenesis techniques.

According to another preferred embodiment, the present invention related to the spinach plant, wherein the resistance gene comprises at least one of the sequences of SEQ ID No. 13, SEQ ID No. 14, and SEQ ID No. 15. Said sequences comprise the one or more mutations providing resistance to Peronospora farinosa. Preferably at least two, most preferably all three of SEQ ID No. 13, SEQ ID No. 14, and SEQ ID No. 15. are present in the spinach plant that is resistant to downy mildew.

The present invention, according to a second aspect, relates to seed produced by a spinach plant according to present invention.

The present invention, according to a third aspect, relates to a resistance gene that confers resistance to downy mildew in spinach plants, wherein the gene encodes for a protein that has at least 85% sequence identity with SEQ ID No. 2, preferably at least 90%, more preferably at least 95%, even more preferably at least 98%, most preferably 99%. The novel resistance gene encodes for a protein that confer broad Pfs resistance in spinach. The coding sequence of the resistance gene has at least 90%, preferably at least 95%, more preferably at least 98%, even more preferably at least 99%, most preferably 99% sequence identity with SEQ ID No. 2. The majority of disease resistance genes in plants encode nucleotide-binding site leucine-rich repeat proteins, also known as NBS-LRR proteins (encoded by R genes). These proteins are characterized by nucleotide-binding site (NBS) and leucine-rich repeat (LRR) domains as well as variable amino- and carboxyl-terminal domains and are involved in the detection of diverse pathogens, including bacteria, viruses, fungi, nematodes, insects and oomycetes. There are two major subfamilies of plant NBS-LRR proteins defined by the Toll/interleukin-1 receptor (TIR) or the coiled-coil (CC) motifs in the amino-terminal domain and are both involved in pathogen recognition. Most recent identified resistances in spinach were identified from a single dominant gene (NBS-LRR), called Locus 1 which is highly variable. Although many alleles have been identified in many different wild spinach accessions on locus 1, none have been identified on locus 3 until now. Therefore, present resistance gene provides a valuable asset in the need for more diversity of alleles and/or locus, so that more genetic variation can be achieved in commercial hybrids, making it harder for pathogens such as Peronospora farinosa to adapt.

According to a preferred embodiment, the present invention relates to the resistance gene, wherein the gene comprises a coding sequence having at least 90%, preferably at least 95%, more preferably at least 98%, most preferably 99% sequence identity with SEQ ID No. 1.

According to another preferred embodiment, the present invention relates to the resistance gene, wherein the resistance gene provides resistance to at least Peronospora farinosa races Pfs3 to Pfs17 in spinach.

According to yet another preferred embodiment, the present invention relates to the resistance gene, wherein the resistance gene comprises at least one of the sequences of SEQ ID No. 13, SEQ ID No. 14, and SEQ ID No. 15. Preferably at least two, most preferably all three of SEQ ID No. 13, SEQ ID No. 14, and SEQ ID No. 15. are comprised in the resistance gene.

According to a further aspect, the present invention relates to a method for providing a spinach plant that is resistant to downy mildew, wherein the method comprises the steps of introducing one or more mutations in a resistance gene in the genome of a susceptible spinach plant thereby providing the downy mildew resistant spinach plant, wherein the resistance gene encodes for a protein that has at least 85% sequence identity with SEQ ID No. 2.

According to a preferred embodiment, the present invention relates to the method, wherein the coding sequence of said resistance gene having at least 90% sequence identity with SEQ ID No. 1.

According to another preferred embodiment, the present invention relates to the method, wherein the one or more mutations of the resistance gene are achieved by genome editing techniques, CRISPR Cas, or mutagenesis techniques.

According to yet another preferred embodiment, the present invention relates to the method, wherein the resistance gene comprises at least one of the sequences of SEQ ID No. 13, SEQ ID No. 14, and SEQ ID No. 15.

The present invention, according to a further aspect, relates to a method for providing a spinach plant that is resistant to downy mildew, wherein the method comprises the steps of

a) providing a spinach plant comprising the resistance gene of present invention,

b) crossing the spinach plant of step a) with a susceptible spinach plant,

c) optionally, selfing the plant obtained in step b) for at least one time,

d) selecting the plants that are resistant to downy mildew.

According to another preferred embodiment, the present invention relates to the method, wherein the spinach plant is resistant to downy mildew caused by Peronospora farinosa races Pfs3 to Pfs17.

According to a preferred embodiment, the present invention relates to the method, wherein the resistance gene is obtained from deposit number NCIMB 43624. Seeds of Spinacia oleracea plant according to present inventions were deposited on 10 Jun. 2020 at NCIMB Ltd, Ferguson Building, Craibstone Estate Bucksburn, AB21 9YA Aberdeen, United Kingdom.

According to a further aspect, present invention relates to the use of one or more markers for providing a spinach plant that is resistant to downy mildew, wherein said one or more markers is selected from the group consisting of SEQ ID No. 3, SEQ ID No. 4, SEQ ID No. 5 and SEQ ID No. 6, preferably SEQ ID No. 3 and SEQ ID No. 4.

### BRIEF DESCRIPTION OF THE DRAWING

The present invention will be further detailed in the following examples and figures wherein:

FIG. 1: shows quantification of Pfs actin in spinach plants infected with Peronospora farinosa (Pfs12 or Pfs14), after VIGS gene silencing. Resistant spinach plants containing the L3 gene were transient transformed with a L3 VIGS silencing construct or a RFP VIGS silencing construct (negative control). As further controls a resistant spinach plant according to present invention was included that was not transformed with a VIGS construct (“R plant) and a Pfs susceptible spinach plant was included (“S control line OS56”). After VIGS, from these plants RNA was isolated to determine the expression levels of the Pfs actin house keeping gene by qPCR to determine Pfs infection. In case L3 gene expression levels were VIGS silenced in spinach infected with Pfs 12 or Pfs 14, expression levels of Pfs actin increased dramatically. Leaves of the plant that were susceptible to Pfs, showed high transcriptional levels of the Pfs actin house keeping gene, indicating the susceptibility corresponds with silenced L3 gene expression due to VIGS silencing.

### DESCRIPTION OF THE INVENTION

### Examples

### Genemapping of Novel Candidate Dominant Resistance Genes

The identification of a novel candidate dominant resistance gene L3 was obtained by gene mapping of Peronospora farinosa (Pfs) resistance genes in Spinach (S. oleracea). In spinach these novel resistance genes were mapped on locus 3 on chromosome 1 in the spinach genome.

The resistance gene was mapped using a Bulk Segregant Analysis (BSA) approach. The RNA of multiple BC3S1 resistant plants were pooled and compared to a pool of RNA of susceptible plants from the same family. Markers were developed in regions where an increase in number of SNPs was observed. The markers were validated on the BC3S1 population. Once a region of interest (ROI) could be identified and flanked by markers, a fine mapping approach was started.

Flanking markers 2969156 and 3155721 or 1941240 and 460539 were used in an F2 population of ˜3000 samples to identify plants that contain a recombination between the two markers.

| | Marker | Sequence |
|---|---|---|
| SEQ ID No. 3 | 2969156 | AATTCCAGGCTGATGCCTCATCGTTCTCAACTGGTAAATG |
| | | GTAACACCCCAAAGTCCAACTGCCAGCCCACCCAATAGA |
| | | GTGAGCAACTTCTGTGATGTTTCTGAATTTTTGTGGCCAA |
| | | TT |
| SEQ ID No. 4 | 3155721 | CATGATTATTGCATAAATTGGGGTTAGGGGTATGATTAA |
| | | TTGCCCCAGTAACAACATATTTGGGAATGTTTTTGGTGTT |
| | | ATTTTGAATGAATGAGGTAGTGGGGGTGGAGGGGGAGA |
| | | AGGATGAGTAAGGTTTGGGAGTGGAGGAGAGAGAAAGA |
| | | GGTGGGTGACGGCGGTGGTGAGGTTTAAGGTGAGACAGT |
| | | GAAGCGG |
| SEQ ID No. 5 | 1941240 | TCACAGTTCATCCCCCTTTGTGTCAAGATGCATGCTATGT |
| | | ATGTGTTCGACGAAATGCCTAAATGAGATGTCAACCGTT |
| | | ATTTCAATTTTGATGCTTAGGTTAGAAGGTGAAATCAAA |
| | | AGA |
| SEQ ID No. 6 | 460539 | GTGTACAAAATTTGGCAGGAAAGGAGCCGACTGAAAAT |
| | | GGTACTGCTAAAAAAATGAGTCAATTTTTAAGCAAGGGG |
| | | CCCATCGTTCTTAACTTTAGAGAATGTTCCCCAGAGGCAG |
| | | ATGA |

Those recombinant plants were phenotyped with several Peronospora farinosa strains and genotyped by markers in the ROI. By combining the genotype and phenotype results, the region of interest could be reduced to a single gene.

Construction of VIGS Construct and Transformation into Spinach (S. olercea)

To confirm if the L3 gene is responsible for the observed resistance in spinach, VIGS silencing can be used to silence the in the resistant source S. oleracea. Therefore, a VIGS-construct was made for L3 and cloned in the K20 vector (See Table 1 for sequences). Another VIGS-construct was made that targets a different gene (RFP) and used as a negative control. The constructs were transformed into spinach using co-cultivation with agrobacterium (GV3101) to study the function of L3 in respect of resistance to Pfs.

TABLE 1

| VIGS-constructs | Sequence |
|---|---|
| L3 (SEQ ID No. 7) | ATGGATGTGTCTGCAGGCCTTTCACTAGTACAAACTGTCCTCG |
| | AATTGTTGGGTTCTCCCATTTGGACACAGATTCAATCTTTGTTG |
| | GGTGTGGAGTACTCACAGCTTGACAAACTCAAGGCCACCATGT |
| | CCACTATTGAGGCTGTGCTCCTTGATGCTGAAGATCAAGAGCA |
| | GTTGCATCAATATCGTCGTAGCCATG |
| RFP (SEQ ID No. 8) | GAGTTCATGCGCTTCAAGGTGCGCATGGAGGGCTCCGTGAACG |
| | GCCACAGTTCGAGATCGAGGGCGAGGGCGAGGGCCGCCCCTA |
| | CGAGGGCACCCAGACCGCCAAGCTGAAGGTGACCAAGGGCGG |
| | CCCCCTGCCCTTCGCCTGGGACATCCTGTCCCCTCAGTTCCAGT |
| | ACGGCTCCAAGGCCTACGTGAAGCACCCCGCCGACATCCCCGA |
| | CTACTTGAAGCTGTCCTTCCCCGAGGGCTTCAAGTGGGAGCGC |
| | GTGATGAACTTCGAGGACGGCGGCGTGGTGACCGTGACCCAG |
| | GACTCCTCCCTGCAGGACGGCGAGTTCATCTACAAGGTGAAGC |
| | TGCGCGGCACCAACTTCTCCTCCGACGGCCCCGTAATGCAGAA |
| | GAAGACCATGGGCTG |

### L3 Resistance Gene Silencing Experiment Using Virus Induced Gene Silencing (VIGS)

To demonstrate that the L3 gene is related to Peronospora farinosa resistance, the putative resistance gene was been silenced by tobacco rattle virus (TRV)-based virus-induced gene silencing (VIGS) to induce susceptibility to P. farinosa infection in resistant spinach lines containing the L3 resistance gene. TRV-derived VIGS vectors have been abundantly described to study gene function in Arabidopsis thaliana, Nicotiana benthamiana, Lycopersicon esculentum and other plants (see for example Huang C, Qian Y, Li Z, Zhou X.: Virus-induced gene silencing and its application in plant functional genomics. Sci China Life Sci. 2012; 55(2):99-108).

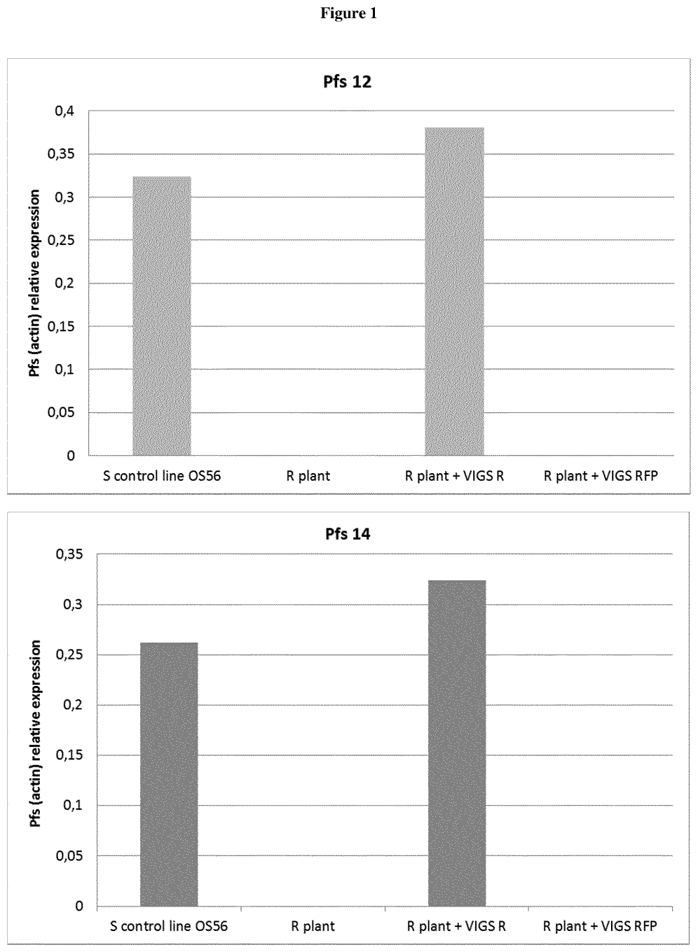

VIGS gene silencing was used to obtain Peronospora-susceptibility in resistant spinach species (S. oleracea) comprising the L3 gene. Briefly, lines containing the L3 gene were silenced by VIGS. Resistant spinach plants were transient transformed with a L3 gene silencing construct and infected with Peronospora farinosa (Pfs 12 or Pfs 14) causing downy mildew in spinach. With VIGS it was demonstrated that the L3 gene was associated with downy mildew resistance, i.e. resistant spinach lines were made susceptible by removing the L3 gene via virus induced gene silencing thereby silencing the L3 resistance gene (see FIG. 1).

Determine Peronospora farinosa Expression in Spinach Comprising the L3 Gene

A qPCR experiment was conducted in spinach tissues obtained from the VIGS experiment as outlined above to determine Peronospora farinosa expression levels in these plants. To obtain more insight in the response of spinach to infection with Peronospora farinosa, leaves of resistant plants comprising the L3 allele, plus the control plants as indicated above, were harvested. cDNA was synthesized from RNA that had been isolated from infected leaves. Expression of Peronospora farinosa actin was analysed by qPCR using the primers as set out in Table 2. (SEQ ID No.9, SEQ ID No.10, SEQ ID No.11, SEQ ID No.12, respectively).

TABLE 2

| Primer name | Sequence |
|---|---|
| P.farinosa actin | 5′-CTCCCCTCAACCCTAAAGCAA-3′ |
| Fwd (SEQ ID No. 9) | |
| P.farinosa actin | 5′-GACAGGACAGCTTGGATGTTTAC-3′ |
| Rv (SEQ ID No. 10) | |
| Elongation factor | 5′-GCAGGGTCGTTCTTTGAGTC-3′ |
| Fwd (SEQ ID No. 11) | |
| Elongation factor | 5′-AGAGGCTCTTCCTGGTGACA-3′ |
| Rv (SEQ ID No. 12) | |

FIG. 1 shows the results of a qPCR of housekeeping gene P. farinosa actin in the plants after Pfs12 or Pfs 14 infection and VIGS silencing. Three technical replicates were performed, and relative Pfs actin expression was analysed by calculating the relative quantity (RQ=1/(2\hat{ }Cttarget)) and normalised expression (NE=RQtarget/RQref). The relative quantity of the target genes were normalised to Elongation factor of Spinacea oleracea, which is a house keeping gene in spinach. Values on the y-axis are relative Pfs actin expression. On the x-axis from left to right: sample leaves of a susceptible spinach plant OS56 in which no VIGS silencing construct has been used, sample leaves of a Pfs resistant plant according to present invention comprising the L3 gene in which no VIGS silencing construct has been used, sample leaves of a plant of present invention in which the L3 gene is silenced using the VIGS silencing construct, and sample leaves of a plant wherein the RFP VIGS construct (negative control) was used. In the samples showing a resistant phenotype, comprising the L3 gene, there is no Pfs present. In the sample with susceptible phenotypes where L3 gene was silenced by VIGS, high transcription levels of the Pfs housekeeping gene actin were measured.

Disease Resistance Test (Leaf Disc Test) for Peronospora farinosa in Spinach L3

Spinach plants of present invention containing the L3 gene are tested for resistance to Peronospora farinosa. The plants must be, at least in the second leaf stage and not yet flowering. Leaves of spinach plants that comprise the L3 gene, were put in trays with moistened paperboard. The infected seedlings are suspended in 20 mL water, filtered by cheesecloth and the flow-through is collected in a spray flask. One tray is spray-inoculated with this Peronospora farinosa suspension. Spray leafs with inoculum and make sure that all the discs are wet. The trays are covered with a glass plate and stored in a climate chamber at 15° C. (12 hours of light). Seven to fourteen days post inoculation infection leaves are phenotypically scored by eye on the presence of Peronospora farinosa (Pfs).

Spinach plants that comprise the L3 gene were tested for resistance phenotype for Pfs. The leaves are scored based on symptoms of sporulation on upper or underside (abaxial side) of the leaf disc and scored according to the following scale:
9=No sporulation.
7-8=A small amount of sporulation (max 10 conidiophores).
5-6=Some sporulation on the edge of the disc.
3-4=Some sporulation in a small area of the punch or many sporulation on a piece of only 2-3 mm close to the edge.
2=Reasonable sporulation.
1=Strong sporulation.
   The infection was validated by inclusion of the susceptible and resistant controls (Viroflay, Boeing, wherein the susceptible control score a score of 1. A score of 9 showed to be fully resistant. Furthermore the degree of sporulation is qualified by the amount of sporulation and not the discoloration of the disc.

TABLE 3
Peronospora farinosa (Pfs) resistance phenotype spinach plants.

| Pfs-physio | Viroflay | Boeing | Plant L3 |
|---|---|---|---|
| 1 | 1 | 9 | ND |
| 2 | 1 | 9 | ND |
| 3 | 1 | 9 | 9 |
| 4 | 1 | 9 | 9 |
| 5 | 1 | 9 | 9 |
| 6 | 1 | 9 | 9 |
| 7 | 1 | 9 | 9 |
| 8 | 1 | 1 | 9 |
| 9 | 1 | 9 | 9 |
| 10 | 1 | 1 | 9 |
| 11 | 1 | 9 | 9 |
| 12 | 1 | 1 | 9 |
| 13 | 1 | 9 | 9 |
| 14 | 1 | 1 | 9 |
| 15 | 1 | 9 | 9 |
| 16 | 1 | 9 | 9 |
| 17 | 1 | 1 | 9 |

Table 3 shows an overview of the disease test performed with the isolates of Peronospora farinosa Pfs1 to Pfs17 on Spinach varieties. Results show that spinach comprising the L3 resistance gene is resistant to at least Peronospora farinosa races Pfs3 to Pfs17. For Pfs physios 1 and 2 resistance was not determined, but it is expected that the spinach plant will also be resistant to Pfs 1 and 2. The control lines show to be susceptible to at least multiple downy mildew isolates. Only the plant of present invention is resistant to the recent physio Pfs 17.

1. A spinach plant that is resistant to downy mildew caused by Peronospora farinosa (Pfs), wherein the spinach plant comprises in its genome a resistance gene comprising one or more mutations, wherein said resistance gene is associated with markers of SEQ ID No. 3 and SEQ ID No. 4 or SEQ ID No. 5 and SEQ ID No. 6.

2. The spinach plant according to claim 1, wherein said resistance gene encodes for a protein having at least 85% sequence identity with SEQ ID No. 2.

3. The spinach plant according to claim 1, wherein said resistance gene comprises a coding sequence having at least 90% sequence identity with SEQ ID No. 1.

4. The spinach plant according to claim 1, wherein the plant is heterozygous or homozygous for the resistance gene comprising one or more mutations.

5. The spinach plant according to claim 1, wherein said plant is at least resistant to Peronospora farinosa races Pfs3 to Pfs17.

6. The spinach plant according to claim 1, wherein said resistance gene is obtainable from deposit number NCIMB 43624.

7. The spinach plant according to claim 1, wherein the one or more mutations comprise deletions, insertions or substitutions in the coding sequence of SEQ ID No. 1.

8. The spinach plant according to claim 1, wherein the resistance gene comprises at least one of the sequences of SEQ ID No. 13, SEQ ID No. 14, and SEQ ID No. 15.

9. A see produced by a spinach plant according to claim 1.

10. A resistance gene comprising one or more mutations that confer resistance to downy mildew in spinach plants, wherein the resistance gene encodes for a protein that has at least 85% sequence identity with SEQ ID No. 2.

11. The resistance gene according to claim 10, wherein the coding sequence of said resistance gene having at least 90% sequence identity with SEQ ID No. 1.

12. The resistance gene according to claim 10, wherein the resistance gene provides resistance to at least Peronospora farinosa races Pfs3 to Pfs17 in spinach.

13. The resistance gene according to claim 10, wherein the resistance gene comprises at least one of the sequences of SEQ ID No. 13, SEQ ID No. 14, and SEQ ID No. 15.

14. A method for providing a spinach plant that is resistant to downy mildew, wherein the method comprises the steps of introducing one or more mutations in a resistance gene in the genome of a susceptible spinach plant thereby providing the downy mildew resistant spinach plant, wherein the resistance gene encodes for a protein that has at least 85% sequence identity with SEQ ID No. 2.

15. The method according to claim 14, wherein the coding sequence of said resistance gene having at least 90% sequence identity with SEQ ID No. 1.

16. The method according to claim 14, wherein the one or more mutations of the resistance gene are achieved by genome editing techniques, CRISPR Cas, or mutagenesis techniques.

17. The method according to claim 14, wherein the resistance gene comprises at least one of the sequences of SEQ ID No. 13, SEQ ID No. 14, and SEQ ID No. 15.

18. The method according to claim 14, wherein the method comprises the steps of:
a) providing a spinach plant comprising the resistance gene,
b) crossing the spinach plant of step a) with a susceptible spinach plant,
c) optionally, selfing the plant obtained in step b) for at least one time,
d) selecting the plants that are resistant to downy mildew.

19. The method according to claim 14, wherein the spinach plant is resistant to downy mildew caused by at least Peronospora farinosa races Pfs3 to Pfs17.

20. The method according to claim 14, wherein the resistance gene is obtainable from deposit number NCIMB 43624.

21. (canceled)


SEQUENCE LISTING (15 sequences)

SEQ ID NO: 1 - DNA, 4608 bp, Spinacia oleracea
atggatgtgtctgcaggcctttcactagtacaaactgtcctcgaattgttgggttctcccatttggacacagattcaatctttgttgggtgtggagtactcacagcttgacaaactcaaggccaccatgtccactattgaggctgtgctccttgatgctgaagatcaagagcagttgcatcaatatcgtcgtagccatgttgaactggataggcttcagaggcttaaggaagctctttatatagctgatgacttgttcgacaagattgccactcttgctcaacgcaaaaccctcataatgcgctttaataacaagctgtttaatgaggtatgccttttcttctcttcctctaataagcttcgttctgctttcacttggtctcgtgagatggagaacattagggagatgttgaatgatattgtgaaagatcatcgctatgactttggctccagacaccccattagtctggctgcgaaacagctgagaatcaaagcaagggaaactcattcctttgtttgcgaggaagatatgatcattgggagggataatgacaagcggaaagtgatagatatgcatagtggggattgggggctgggaaaaacaactctagcccgacttgtttataatgatgagaagataggtagtaaattctctctgaaaatgtgggtttgtgtctctgatagtttcaatgtcaaagatcttgttgctgaaatccttgcagccgtaaaccctagaagaaagcacgatggcctgagtatgagtatggaccagcttcagatgaaacttcgggacgaactggatgggaagatatacctccttgttttggatgatgtatggactgaagatcccattaaatggcatgagttgagaaaactattgattggaggtagaaacggtagcagaattctggtgactactcgctcaagtggggtggcgaaagttgttggaagctatcatattcataaattggaaggactctctaataaagagtcttgggatttgtttgagaggatgacacttgaaccaggacaacatcaaatgcaagagcacttggttaaaattggaaaagatattgtgagaaagtgtgcaaatgttccacttgctataagggtggttggaagtcttcttcgaggtcagggtgaaagtaggtggcaatatttgaaaaacactgacttggcaaatataccacaagatgagatcaatggtattttacctgttttgaagattagttattattatcttccgttccatttgaagagttgctttaagtattgttctgtattccccaaggattacaagattatcaaggaggatttgatcagtctctggatggcacaaggcttcatcatctcatcaaatgaagagagtttcgaaaatgcaggtgaggaatatttcaaacaattactccaaaggtgtttcttccaagatgtagaacgggccgagggtactgatgaaattctatcatgcaagatgcatgacttgattcatgaccttgctagtgaagtagcaggaacagaaatcgtgtcgtccaagtataatataagggggtttagcgagaaaactcgccatatatttattgatagagatgccgttgaagatatttatcgtcatttcaccaatatgaaaaggatgcgttcaatttttagaatagatgcgctttcttctaattcaacaaatttgttatcaaaagtggaatatttaagggtcttaagcttgaataactctggtttggaaatgttgccgagtgagatagacgcagttgatgctagggaaagggggtttctattaaaaagtcaacatttgcgggaaattcggtatgtttggaatcatgagagtgaggggactgatgcagatgcattgctgcaaggaaaaaatgcagatgtagtgctgcaagggctgcaaccccatcctaatctccgaatgctggagttgagggattatccaggaattggattcccaagctgggggaggtcatcaatgaatctccatacttgtctcccaaatcttgttagcataacactatatggatgtacaaggttggagcatcttccattgatgagtcaactgcgtcatttgaaattccttacattacgatatttgaatgaagtgctcctaactatctacaagtataactttgttcatggtataagtgcagaagacgaagttgaggccctccaatccttgcaatttgaccttgaaactatagaaattgcaacacataacttttctgatgataataagatcggagaaggcggattcggcccggtttataagggtatacttcctgatgggcaagagatagcagttaagaggctttataggaactcgcgccaaggagatgcagagttcaaaaatgaaattttgatattagccaaggtccggcacaagaatttggtgaaacttctgggattttgcttgcatggagaagaaaggattcttatccttgagtttgttgccaacagaagccttgacaactttatatttgatccattgagtcgtcagtctatgcaatgggaaactcgttataagattatcaatggcatagctagagggattctatacctccatgaaggttccagactaatgatcatacatcgcaacctaaaagcaggaagtgttctacttgatgaagatttttgtcccaagattgcagattttggtatggcatggctatttaacatagatcaaactcaaacagagactagcacaatcgctggaacctttggatatatggcacctgagtacgctgagcaaagtcaagtatcagtaaagttagacgtatacagctttggtgtactagtcctagagatagcttggagaaactggaatgaaggcacagctcggaatctggtagatcctgtgttactagccggctctagtaccgagatactgcgatacatccatatcggtttgttgtgtgtccaagaaagccctgcagacaggccaactatgtctgaggttcatgttatgttgagtacgaattcggtcacctttgaagcacctttgcagcctgcattttatacagatagccaaagtctacccgatatgcttccggagtggagcacaactaatagtcaagtatcatatgatgaatcagagccggagtggagtcctctcctctcaatgaatttctccgataaaatggattcgccggacgtcatctcctctcgccgccaaccttcaaccttcgtcggcgaaaatggtggattcgccaaactttccaccacctgcaccaccaaattaccggaagaagcagcttgtatttctggttcatcaaaacctacttcccgacttcaaatcaattgcacaattcgaaagttaagggttgaggttttgcgcgatatgttggggaatgcgttgaaaccggattttttagcatacacgaccttgttggaggagatgtgtagggatggacagggtgttgatgcattagagcttctcgaggagtttaggaagacggatgtatcgatgggggaaaaggcttatagaactttgttggatacattgcatttcgtggatcggtttcaaggtacatggattaggaactggatgatacaaaatttgccccaaaagctttgtagaattgcaggtgcaaatggaagtcccttttggacggtgaaggtgggaagaagaaatgggcagcctaaactggaagaggaatcgagggagtggaagaggaaacgtgggagagagaaaaagaaatgggttcccaagatgcctggtggtggcggcggcgctgctgctaatttggttaaggttggaattcttggtgctcttggccttggcgcttatggagtcagtcatagtatgtacaatgttgaaggtggtcatcgagctatcgtcttcaaccgcatctctggtatcaaggacaaggtgtaccctgaaggaacacatctcatgattccatggctcgagagaccaatcatttatgatgtccgtgcacgacctcatctagtagaaagtacatctggaagtcgtgatcttcaaatggcccgccctgaaaaatatgaagtggatgggtgtagggtcaagattggccttcgagttcttactcgccctttaccagaaaccttgccaacaatttaccgtacacttggtgaaaattacagtgaaagagtcttgccatccattgtccatgaaacccttaaagccgtggttgcccaatacaatgcaagtcagcttcttactcaacgtgaggctgttagtagggagattcgaaaagtattgacagaaagagcttcaaacttcaacattgctttggatgatgtgtctatcaccactcttaactttggtagagagttcactgctgcaattgaggctaaacaggtagctgcacaagatgctgaaagagccaagtttattgtggaaaaggctgagcaagacaagaaaagtgctatcatcagagctcagggagaagccaagagtgctcaacttattggtcaagcaatttccaacaacccggcttttattactctgagaagaatcgaagcagccagggaaatcgcccatgtaatctctaattcagcaaacaaggtgatgttgagttcggacgatcttctcctcaatcttggtgatatgacaccagatgcacctattgcaaagaagtaa

SEQ ID NO: 2 - protein, 1535 aa, Spinacia oleracea
MDVSAGLSLVQTVLELLGSPIWTQIQSLLGVEYSQLDKLKATMSTIEAVLLDAEDQEQLHQYRRSHVELDRLQRLKEALYIADDLFDKIATLAQRKTLIMRFNNKLFNEVCLFFSSSNKLRSAFTWSREMENIREMLNDIVKDHRYDFGSRHPISLAAKQLRIKARETHSFVCEEDMIIGRDNDKRKVIDMHSGDWGLGKTTLARLVYNDEKIGSKFSLKMWVCVSDSFNVKDLVAEILAAVNPRRKHDGLSMSMDQLQMKLRDELDGKIYLLVLDDVWTEDPIKWHELRKLLIGGRNGSRILVTTRSSGVAKVVGSYHIHKLEGLSNKESWDLFERMTLEPGQHQMQEHLVKIGKDIVRKCANVPLAIRVVGSLLRGQGESRWQYLKNTDLANIPQDEINGILPVLKISYYYLPFHLKSCFKYCSVFPKDYKIIKEDLISLWMAQGFIISSNEESFENAGEEYFKQLLQRCFFQDVERAEGTDEILSCKMHDLIHDLASEVAGTEIVSSKYNIRGFSEKTRHIFIDRDAVEDIYRHFTNMKRMRSIFRIDALSSNSTNLLSKVEYLRVLSLNNSGLEMLPSEIDAVDARERGFLLKSQHLREIRYVWNHESEGTDADALLQGKNADVVLQGLQPHPNLRMLELRDYPGIGFPSWGRSSMNLHTCLPNLVSITLYGCTRLEHLPLMSQLRHLKFLTLRYLNEVLLTIYKYNFVHGISAEDEVEALQSLQFDLETIEIATHNFSDDNKIGEGGFGPVYKGILPDGQEIAVKRLYRNSRQGDAEFKNEILILAKVRHKNLVKLLGFCLHGEERILILEFVANRSLDNFIFDPLSRQSMQWETRYKIINGIARGILYLHEGSRLMIIHRNLKAGSVLLDEDFCPKIADFGMAWLFNIDQTQTETSTIAGTFGYMAPEYAEQSQVSVKLDVYSFGVLVLEIAWRNWNEGTARNLVDPVLLAGSSTEILRYIHIGLLCVQESPADRPTMSEVHVMLSTNSVTFEAPLQPAFYTDSQSLPDMLPEWSTTNSQVSYDESEPEWSPLLSMNFSDKMDSPDVISSRRQPSTFVGENGGFAKLSTTCTTKLPEEAACISGSSKPTSRLQINCTIRKLRVEVLRDMLGNALKPDFLAYTTLLEEMCRDGQGVDALELLEEFRKTDVSMGEKAYRTLLDTLHFVDRFQGTWIRNWMIQNLPQKLCRIAGANGSPFWTVKVGRRNGQPKLEEESREWKRKRGREKKKWVPKMPGGGGGAAANLVKVGILGALGLGAYGVSHSMYNVEGGHRAIVFNRISGIKDKVYPEGTHLMIPWLERPIIYDVRARPHLVESTSGSRDLQMARPEKYEVDGCRVKIGLRVLTRPLPETLPTIYRTLGENYSERVLPSIVHETLKAVVAQYNASQLLTQREAVSREIRKVLTERASNFNIALDDVSITTLNFGREFTAAIEAKQVAAQDAERAKFIVEKAEQDKKSAIIRAQGEAKSAQLIGQAISNNPAFITLRRIEAAREIAHVISNSANKVMLSSDDLLLNLGDMTPDAPIAKK

SEQ ID NO: 3 - DNA, 121 bp, Artificial Sequence
  Marker sequence
aattccaggctgatgcctcatcgttctcaactggtaaatggtaacaccccaaagtccaactgccagcccacccaatagagtgagcaacttctgtgatgtttctgaatttttgtggccaatt

SEQ ID NO: 4 - DNA, 201 bp, Artificial Sequence
  Marker sequence
catgattattgcataaattggggttaggggtatgattaattgccccagtaacaacatatttgggaatgtttttggtgttattttgaatgaatgaggtagtgggggtggagggggagaaggatgagtaaggtttgggagtggaggagagagaaagaggtgggtgacggcggtggtgaggtttaaggtgagacagtgaagcgg

SEQ ID NO: 5 - DNA, 121 bp, Artificial Sequence
  Marker sequence
tcacagttcatccccctttgtgtcaagatgcatgctatgtatgtgttcgacgaaatgcctaaatgagatgtcaaccgttatttcaattttgatgcttaggttagaaggtgaaatcaaaaga

SEQ ID NO: 6 - DNA, 121 bp, Artificial Sequence
  Marker sequence
gtgtacaaaatttggcaggaaaggagccgactgaaaatggtactgctaaaaaaatgagtcaatttttaagcaaggggcccatcgttcttaactttagagaatgttccccagaggcagatga

SEQ ID NO: 7 - DNA, 199 bp, Artificial Sequence
  VIGS construct
atggatgtgtctgcaggcctttcactagtacaaactgtcctcgaattgttgggttctcccatttggacacagattcaatctttgttgggtgtggagtactcacagcttgacaaactcaaggccaccatgtccactattgaggctgtgctccttgatgctgaagatcaagagcagttgcatcaatatcgtcgtagccatg

SEQ ID NO: 8 - DNA, 400 bp, Artificial Sequence
  VIGS construct
gagttcatgcgcttcaaggtgcgcatggagggctccgtgaacggccacagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttctcctccgacggccccgtaatgcagaagaagaccatgggctg

SEQ ID NO: 9 - DNA, 21 bp, Artificial Sequence
  Forward primer
ctcccctcaaccctaaagcaa

SEQ ID NO: 10 - DNA, 23 bp, Artificial Sequence
  Reverse primer
gacaggacagcttggatgtttac

SEQ ID NO: 11 - DNA, 20 bp, Artificial Sequence
  Forward primer
gcagggtcgttctttgagtc

SEQ ID NO: 12 - DNA, 20 bp, Artificial Sequence
  Reverse primer
agaggctcttcctggtgaca

SEQ ID NO: 13 - DNA, 90 bp, Spinacia oleracea
tagcgagaaaactcgccatatatttattgatagagatgccgttaaagatatttatcgtcatttcaccaatatgaaaaggatgcgttcaat

SEQ ID NO: 14 - DNA, 63 bp, Spinacia oleracea
agatgcagttgatgctagggaaaaggggtttctattaaaaagtgaacatttgagggaaattca

SEQ ID NO: 15 - DNA, 85 bp, Spinacia oleracea
gaaaaatgcagatgtggtgctgcaagggctgcaaccccatcttaatctccgaatgttggagttgagggattatccaggaattgga